(19) United States
(12) Patent Application Publication
(10) Pub. No.: US 2013/0083290 A1
(43) Pub. Date: Apr. 4, 2013

(54) OPHTHALMOLOGICAL ANALYSIS METHOD

(71) Applicant: Palol Vinciguerra, Milano (IT)

(72) Inventor: Palol Vinciguerra, Milano (IT)

(21) Appl. No.: 13/629,484

(22) Filed: Sep. 27, 2012

(30) Foreign Application Priority Data
Sep. 29, 2011 (DE) 102011083789.2

(51) Int. Cl. A61B 3/107

(52) U.S. Cl. 351/246

(57) ABSTRACT

An ophthalmological analysis method for measuring a curvature of the cornea of an eye with an analysis system consists of a measuring device with which topographical data of the cornea is calculated, and an analysis device with which a curvature (r1, r2) of the cornea is derived from the topographical data of the cornea, wherein a curvature gradient of the cornea is derived from the corneal topography data using the analysis device.

### CROSS REFERENCE TO RELATED APPLICATION

The present application claims the benefit of German Patent Application No. 10 2011 083 789.2 filed Sep. 29, 2011, which is fully incorporated herein by reference.

### STATEMENT REGARDING FEDERALLY SPONSORED RESEARCH OR DEVELOPMENT

Not applicable.

### FIELD OF THE INVENTION

The invention relates to an ophthalmological analysis method for measuring a curvature of an eye cornea using an analysis system consisting of a measuring device that is used to determine corneal topography data and an analysis device that is used to derive a curvature of the cornea from the corneal topography data.

Analysis systems of such kind are sufficiently known from the prior art, and the measuring devices used in each may be based on an extremely wide variety of measuring methods. With the known measuring devices, it is possible to determine at least a topography of an outer surface of an eye cornea. The topography data determined in this context is often analysed and processed further with an analysis device of the analysis system, wherein a curvature of the cornea and other information may be determined at a point or region of the corneal surface from the topography data. It should be noted that a curvature or radius of curvature of the cornea is essentially always constant over the surface of the cornea unless a deformation such as an aberration caused by the cornea, keratokonus or corneal damage is present.

Besides wearing spectacles or contact lenses, methods used to correct vision defects or the refractive strength of an eye include refractive surgery. Lower order aberrations can be corrected by using glasses or contact lenses, and higher order aberrations can be corrected surgically, for example by using laser-assisted in-situ keratomileusis (LASIK). In this laser method, the curvature of the cornea is modified by removing tissue from within the cornea. This ablation of tissue in the cornea is enabled by cutting and opening the cornea. Other refractive surgery methods, such as PRK, LASEK or epi-LASEK provide for treatment of the surface of the cornea. In all these methods, the cornea is thinned by ablating the tissue and thus also modifying the curvature of the cornea in this area. As has been found, however, over extended periods the cornea tends to smooth out or compensate for such modifications of a curvature or discontinuities in curvature through cell growth or replacement. Thus for example, the visual acuity of an eye may be altered demonstrably following a surgical procedure over a period of two years, for example. A change in visual acuity in this context always depends on a modification or discontinuity of the curvature in the area of the surface of the cornea. A change in visual acuity or a modification of curvature may, inter alia, also be caused by a cataract, corneal transplant, vitrectomy, glaucoma, corneal oedema or bacterial abscess, etc. It would therefore be advantageous to be able to quantify a discontinuity or curvature in the area of the surface of an eye cornea more precisely, in order to be able to better estimate any possible change in visual acuity following a surgical procedure and a change in the curvature of the cornea for a future period.

### SUMMARY OF THE INVENTION

The task underlying the present invention is therefore to suggest an ophthalmological analysis method with which a change in the curvature of the cornea may be measured. This task is solved by a method including the step of deriving a curvature gradient of the cornea from corneal topographical data using an analysis device.

The ophthalmological analysis method according to the invention for measuring a curvature of an eye cornea is performed with an analysis system, wherein the analysis system consists of a measuring device and an analysis device, wherein topography data of the cornea is determined with the measuring device and a curvature of the cornea is derived from the corneal topography data with the analysis device, wherein a curvature gradient of the cornea is derived from the corneal topography data with the analysis device.

In particular, the determination of a curvature gradient enables a discontinuity in the curvature of the surface of the cornea of the eye to be described with a quantifiable, measurable value. It also becomes possible to determine the curvature gradient for various points on the surface of the cornea, so that a geometrical characterisation or position of a discontinuity in the curvature may be determined. Any change in visual acuity in the future after a surgical procedure or any change in visual acuity caused in another way may be estimated from the measured curvature gradient and its position. It is particularly advantageous that the curvature gradient is simply derived from the topographical data of the cornea, which is calculated in any case during a various eye examinations. While it is true that a number of different curvature radii of a cornea could be calculated if necessary from previously known topography measurements, it was previously not possible to describe a transition zone between the curvature radii in more precisely measurable terms. In particular, in this case it is now possible to make a statement about the magnitude of a change in curvature. At the same time, the way in which the curvature gradient of the cornea is derived from the topography data is generally not important for performing the method at first.

In an advantageous embodiment of the method, the topographical data may include a plurality of topography dataset, each of which describes a point on the cornea surface, wherein the point may definable by its position in a three-dimensional coordinate system in the manner of a spatial model. For example, each point on the cornea surface may be described simply relative to the coordinate system in the form of a topography dataset. For example, an X, Y and Z coordinate may be specified for each point, so that a spatial model of the cornea surface may be derived from the topographical data.

In a further process step, a first differential quotient may be calculated from points with the analysis device, in which process a gradient may be determined within each point in question. In this way, it is possible to determine a gradient for all points of a three-dimensional spatial model of the cornea surface.

A second differential quotient of the points may also be calculated with the analysis device, wherein one curvature or curvature radius may be determined for each point. In this way, a radius of curvature may be determined for each point in the three-dimensional spatial model of the cornea surface in similar manner to the gradient. Accordingly, it thus becomes possible even now to determine differences between various radii of curvature. Thus the regions of the cornea in which the surgical procedure was carried out and which are presenting corneal deformation may be identified even at this stage of the method.

Finally, a third differential quotient of the points may be calculated with the analysis device, wherein a curvature gradient or gradient of the radius of curvature may be determined for each point. Thus a direction may be determined for the gradient of curvature as well as the magnitude thereof. In this way, it is easy to determine the areas of the cornea in which the greatest curvature gradients are located and whether a size of the curvature gradient indicates that a long-term change in visual acuity is likely. In particular, a link between gradient of curvature and higher order aberrations can be determined. Depending on the change in gradient of curvature, the progression of the healing process following treatment can therefore be assessed and the success of the healing process can be quantified. The gradient of curvature can also be used to make a basic distinction between lower order aberrations and higher order aberrations, and therefore may be consulted in order to select a suitable treatment method.

Particularly when a sufficient quantity of topographical data describing a cornea has been collected, a scalar may be created from the topographical data via the analysis device. The scalar field may describe for example a gradient, a radius of curvature, or another measurable property of any point on the surface of the cornea. In this way, it is possible to create an overview of a distribution of the measurable variables over the surface of the cornea.

Thus for example, a visual representation of the scalar field may also be compiled and output via the analysis device. A specialist who is examining the eye is thus able to gain an overview of the measurement data for the eye very easily.

The analysis device may also be used to create a gradient field from the curvature gradients. A gradient field is a vector field that is derived by differentiation according to the site, or a gradient of a scalar field. For example, a rate and direction of change for a change in size of the scalar field may be indicated, with specification of a radius of curvature, for example. In the present case, the gradient of the scalar field may be equivalent to the gradient of curvature of the cornea.

A visual representation of the gradient field may also be compiled and output via the analysis device. In this case too, someone who is analysing the eye according to this method is able to gain a particularly good overview of the distribution of the curvature gradient over the surface of the cornea and a magnitude and direction thereof.

The analysis device may include means for processing data and a database, wherein the database contains datasets of curvature radii and correction values of the curvature gradient assigned to each curvature gradient, wherein the calculated curvature gradients may be assigned together with the curvature gradients stored in the database, wherein the calculated topographical data may be corrected with the respective correction values of matching curvature gradients. To this extent, the database may contain datasets for which the correction values assigned to the curvature gradients are derived from empirical values or comparison measurements. The correction values may thus describe a change in visual acuity over the course of a relatively long period after a surgical procedure in or on the cornea. If matching or similar curvature gradients are present for the eye that is being examined or measured, it may be assumed that an essentially equivalent change in visual acuity is taking place for these curvature gradients.

Accordingly, a future change in visual acuity for the eye being measured may be determined from the corrected topographical data. It thus becomes possible to correct this with regard to an expected visual acuity after or even during a surgical procedure. It is also easily possible to prepare a forecast of a possible change in visual acuity.

It is particularly advantageous if the topographical data is calculated from cross-sectional images of the cornea. In this way, not only the surface of the cornea but also other data describing the cornea, such as corneal thickness, may also be included in the measurement. In particular, by measuring corneal thickness at the same time it is possible to include an intraocular pressure that is normally exerted on the cornea in the calculation. Thus for example, an area where the cornea is thinner than usual may present a bulge due to intraocular pressure and thus also a change in curvature or curvature gradient. Furthermore, a link between curvature gradient and pachymetry measurement may also be determined.

The cross-sectional images of the cornea may be obtained particularly advantageously with a Scheimpflug system consisting of a slit lighting device and an observation device in a Scheimpflug arrangement. In this way, it is possible to capture an entire region of an anterior eye chamber, for example with a camera of the observation device, and to derive and calculate the optical boundary surfaces from the image data obtained. The Scheimpflug system may also be designed so as to be pivotable about a visual axis of the eye, so that a large number of cross-sectional images may be obtained. If the cross-sectional images are obtained for various angles of rotation of the Scheimpflug system relative to the visual axis, a three-dimensional model of the anterior ocular chamber may be derived from the cross-sectional images, and the topographical data of the cornea among other information may be calculated very easily from the three-dimensional model.

Alternatively, it is possible for the topographical data to be calculated using a keratometer. The keratometer may for example be a video-keratograph with a placido ring, or it may perform calculate the topographical data on the basis of a wavefront analysis.

It is also alternatively possible for the topographical data to be calculated by means of optical coherence tomography (OCT). As well as obtaining topographical data, high-resolution three-dimensional microscopy can also be carried out on the living tissue.

A laser device for laser-assisted in-situ keratomileusis (LASIK) may also be operated according to the respective curvature gradients determined. For example, the cornea can be cut particularly precisely during a surgical procedure. The surgical procedure may thus also be carried out in an automated or partly automated manner based on the data established using the analysis method.

### BRIEF DESCRIPTION OF THE DRAWINGS

In the following, an embodiment of the invention will be explained in greater detail with reference to the accompanying drawing. In the drawings:

FIG. 1 shows a diagrammatical view of a cross sectional view of a cornea of an eye along a visual axis.

### DETAILED DESCRIPTION OF THE EXAMPLE EMBODIMENTS

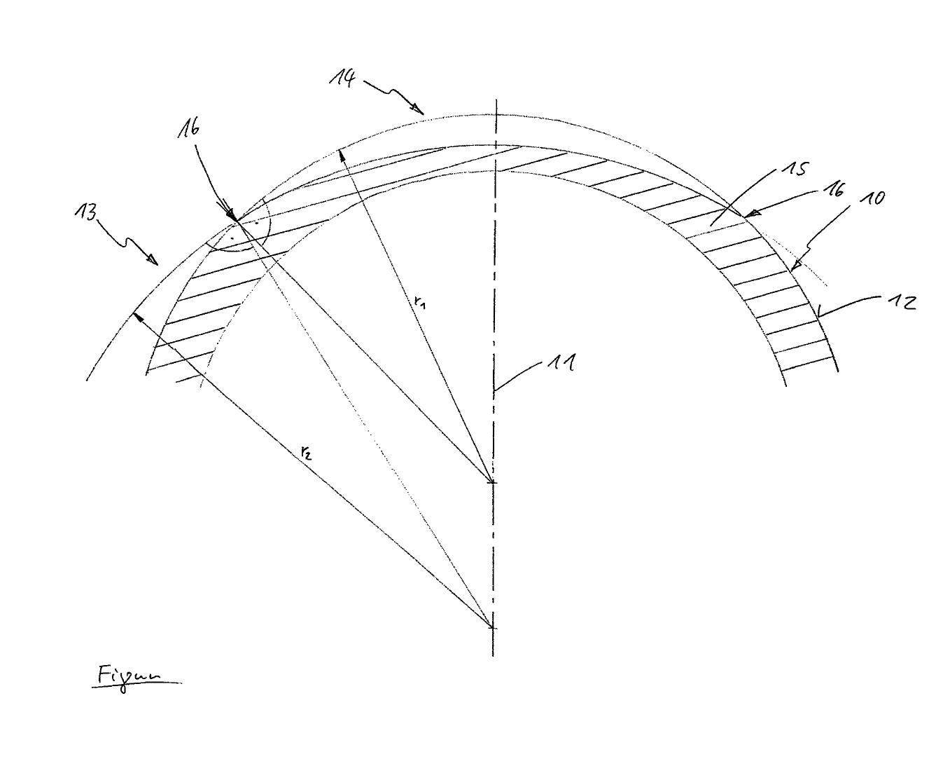

FIG. 1 shows a simplified, diagrammatic representation of a cross sectional view of a cornea 10 of an eye, not shown in greater detail, along a visual axis 11. An outer surface 12 of cornea 10 has a curvature with radius r₁ in a peripheral area 13 of cornea 10. Tissue material 15 of cornea 10 in a central area 14 of cornea 10, indicated by hatching in the figure, was removed in a surgical procedure, so that the cornea 10 in central area 14 thereof has a curvature with radius r₂. r₂ is larger than r₁. In addition, a thickness of cornea 10 is considerably reduced in central area 14 compared with peripheral area 13. As a result, a discontinuity of curvature or a significant change in the curvature gradient occurs in a transition area 16 between curvature radii r₁ and r₂ of the outer surface 12 of cornea 10.

According to the analysis method, the curvature gradient in transition area 16 is determined by calculating a differential quotient or deriving a curvature in transition area 16. On the basis of the calculated curvature gradient, it then becomes possible to estimate a possible change in visual acuity as a result of cellular changes in transition area 16, and thus also a change in the curvature gradient, even just after the surgical procedure is completed.

1. An ophthalmological analysis method for measuring a curvature of the cornea of an eye with an analysis system, said method comprising:
obtaining topographical data of the cornea using a measuring device;
deriving a curvature (r1, r2) of the cornea from the topographical data of the cornea using an analysis device; and
deriving a curvature gradient of the cornea from the corneal topographical data using the analysis device.

2. The analysis method as recited in claim 1, in which the topographical data includes a plurality of topographical datasets, each of which describes one point on a cornea surface, wherein the point is defined by its position in a three-dimensional coordinate system.

3. The analysis method as recited in claim 2, in which a first differential quotient of points is calculated using the analysis device, wherein a gradient is determined for each of the points.

4. The analysis method as recited in claim 3, in which a second differential quotient of points is calculated using the analysis device, wherein a curvature (r1, r2) is determined for each of the points.

5. The analysis method as recited in claim 4, in which a third differential quotient of points is calculated using the analysis device, wherein a curvature gradient is determined for each of the points.

6. The analysis method as recited in claim 1, characterised in that a scalar field is formed from the topographical data using the analysis device.

7. The analysis method as recited in claim 6, in which a visual representation of the scalar field is compiled and output using the analysis device.

8. The analysis method as recited in claim 1, characterised in that a gradient field is created from the curvature gradients using the analysis device.

9. The analysis method as recited in claim 8, in which a visual representation of the gradient field is compiled and output using the analysis device.

10. The analysis method as recited in claim 1, the analysis device includes a database, wherein the database contains datasets of curvature gradients and correction values assigned to the respective curvature gradients, wherein the calculated topographical data is corrected with the respective correction values of matching curvature gradients.

11. The analysis method as recited in claim 10, in which a future change in visual acuity is determined for the eye being measured from the corrected topographical data.

12. The analysis method as recited in claim 1, in which the topographical data is determined from cross sectional images of the cornea.

13. The analysis method as recited in claim 12, in which the cross-sectional images of the cornea are obtained with a Scheimpflug system consisting of a slit lighting device and an observation device in a Scheimpflug arrangement.

14. The analysis method as recited in claim 13, in which the Scheimpflug system is pivotable about an axis of vision of the eye, where a plurality of cross-sectional images is obtained.

15. The analysis method as recited in claim 1, in which the topographical data is determined using a keratometer.

16. The analysis method as recited in claim 1, in which the topographical data is determined using optical coherence tomography.

17. The analysis method as recited in claim 1, in which a laser device for laser-assisted in-situ keratomileusis (LASIK) is operated according to the respective curvature gradients determined.
